(19) United States
(12) Patent Application Publication
(10) Pub. No.: US 2025/0205447 A1
(43) Pub. Date: Jun. 26, 2025

(54) REHABILITATION METHOD AND REHABILITATION DEVICE USING THE SAME

(71) Applicant: INDUSTRIAL TECHNOLOGY RESEARCH INSTITUTE, Hsinchu (TW)

(72) Inventors: Hung-Kai CHEN, Hsinchu City (TW); Ming-Chieh TSAI, Xinpu Township (TW); Tsung-Hua LI, Hsinchu City (TW); YOONG KEE SEK, Hsinchu City (TW)

(73) Assignee: INDUSTRIAL TECHNOLOGY RESEARCH INSTITUTE, Hsinchu (TW)

(21) Appl. No.: 18/397,623

(22) Filed: Feb. 27, 2024

(30) Foreign Application Priority Data
Dec. 22, 2023 (TW) 112150296

(51) Int. Cl. A61M 21/00

(52) U.S. Cl. CPC A61M 21/00; A61M 2021/0027; A61M 2205/056; A61M 2205/3331; A61M 2205/50; A61M 2230/63

(57) ABSTRACT

A rehabilitation device includes a music player, a first pressure sensing device and a processor. The music player is configured to play a rhythm music at a beat speed. The first pressure sensing device is configured to capture a plurality of first foot pressure distributions. The processor is configured to obtain a plurality of first trigger mode pressure distributions that match a trigger mode in the first foot pressure distributions; obtain a trigger speed of the first trigger mode pressure distributions in the first foot pressure distributions; increase the beat speed based on the beat speed being slower than the trigger speed; and reduce the beat speed based on the beat speed being quicker than the trigger speed.

This application claims the benefit of Taiwan application Serial No. 112150296, filed Dec. 22, 2023, the disclosure of which is incorporated by reference herein in its entirety.

### TECHNICAL FIELD

The disclosure relates in general to a rehabilitation method and a rehabilitation device using the same.

### BACKGROUND

A rehabilitation may help patients recover more quickly. However, current rehabilitation programs mostly require the rehabilitation person to perform a repetitive movement which is boring, and also reduces the rehabilitation action of the rehabilitation person. Therefore, proposing a rehabilitation method that may improve the aforementioned problems is one of the goals of those in this technical field.

### SUMMARY

The embodiment of the present invention proposes a rehabilitation method and a rehabilitation device using the same, which may improve the aforementioned conventional problems.

According to an embodiment, a rehabilitation method is provided. The rehabilitation method includes the following steps: playing a rhythm music at a beat speed by a music player; capturing a plurality of first foot pressure distributions by a first pressure sensing device; obtaining a plurality of first trigger mode pressure distributions which meet a trigger mode in the first foot pressure distributions by a processor; obtaining a trigger speed of the first trigger mode pressure distributions in the first foot pressure distributions by the processor; increasing the beat speed based on the beat speed being slower than the trigger speed by the processor; and reducing the beat speed based on the beat speed being faster than the trigger speed by the processor.

According to another embodiment, a rehabilitation device is provided. The rehabilitation device includes a music player, a first pressure sensing device and a processor. The music player is configured to play a rhythm music at a beat speed. The first pressure sensing device is configured to capture a plurality of first foot pressure distributions. The processor is configured to obtain a plurality of first trigger mode pressure distributions which meet a trigger mode in the first foot pressure distributions; obtain a trigger speed of the first trigger mode pressure distribution in the first foot pressure distributions; increase the beat speed based on the beat speed being slower than the trigger speed; and reduce the beat speed based on the beat speed being faster than the trigger speed.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1 illustrates a functional block diagram of a rehabilitation device 100 according to an embodiment of the present disclosure;

FIG. 2 illustrates a schematic diagram of a gait of a rehabilitation person wearing a pressure sensing device 120;

FIG. 3A illustrates a schematic diagram of a plurality of first pressure sensing values PAv of a first foot pressure distribution PA sensed by a first pressure sensing device 120A in FIG. 2;

FIG. 3B illustrates a schematic diagram of a plurality of second pressure sensing values PBv of a second foot pressure distribution PB sensed by a second pressure sensing device 120B in FIG. 2;

FIG. 4 illustrates a schematic diagram of a rehabilitation action of the rehabilitation person matching a rhythm music T1 played by a music player 110 in FIG. 1;

FIG. 5 illustrates a schematic diagram of the maximum extreme values and the beat point T11 represented by R-score map according to an embodiment of the present invention; and

FIG. 6 illustrates a flow chart of the rehabilitation method of the rehabilitation device 100 in FIG. 1.

### DETAILED DESCRIPTION

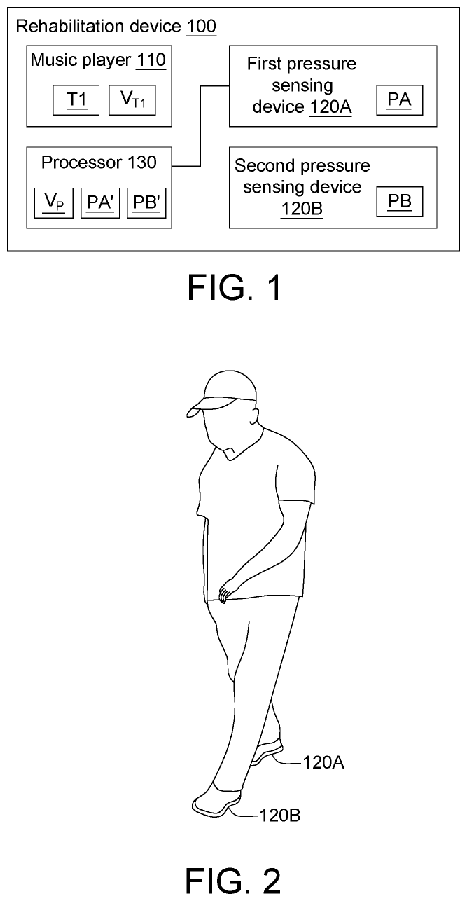

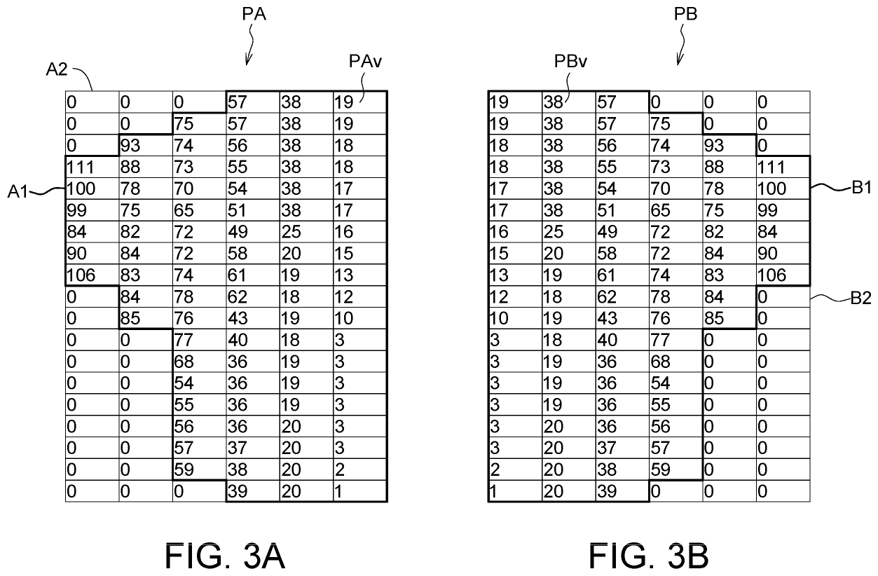

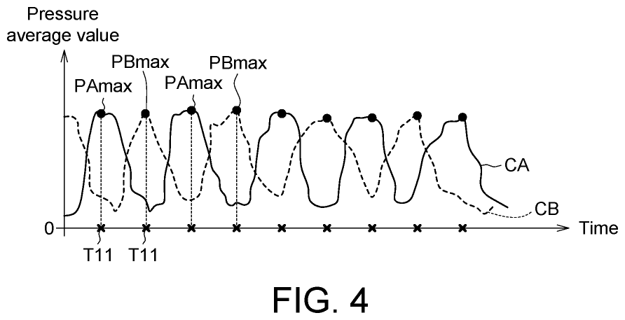

Referring to FIGS. 1 to 4, FIG. 1 illustrates a functional block diagram of a rehabilitation device 100 according to an embodiment of the present disclosure, FIG. 2 illustrates a schematic diagram of a gait of a rehabilitation person wearing a pressure sensing device 120, FIG. 3A illustrates a schematic diagram of a plurality of first pressure sensing values PAv of a first foot pressure distribution PA sensed by a first pressure sensing device 120A in FIG. 2, FIG. 3B illustrates a schematic diagram of a plurality of second pressure sensing values PBv of a second foot pressure distribution PB sensed by a second pressure sensing device 120B in FIG. 2, and FIG. 4 illustrates a schematic diagram of a rehabilitation action of the rehabilitation person matching a rhythm music T1 played by a music player 110 in FIG. 1.

As illustrated in FIG. 1, the rehabilitation device 100 includes the music player 110, the first pressure sensing device 120A, the second pressure sensing device 120B and a processor 130. The music player 110 and/or the processor 130 include physical circuits, for example, a semiconductor chip, a semiconductor package, etc., formed by using, for example, at least one semiconductor process. The music player 110 may be electrically connected to a speaker to output sound, or the music player 110 itself may include the speaker. The first pressure sensing device 120A includes, for example, a plurality of first pressure sensing units. Each first pressure sensing unit may sense the first pressure sensing values PAv (the first pressure sensing values PAv is illustrated in FIG. 3A). The second pressure sensing device 120B includes, for example, a plurality of second pressure sensing units. Each second pressure sensing unit may sense the second pressure sensing values PBv (the second pressure sensing values PBv is illustrated in FIG. 3B).

As illustrated in FIGS. 1, 3A and 3B, the music player 110 is configured to play the rhythm music T1 at a beat speed V_{T1}. The first pressure sensing device 120A captures a plurality of the first foot pressure distribution PA. The second pressure sensing device 120B captures a plurality of the second foot pressure distribution PB. The processor 130 is configured to obtain a plurality of first trigger mode pressure distributions PA′ that meets a trigger mode in the first foot pressure distributions PA; obtain a plurality of second trigger mode pressure distributions PB′ that meet the trigger mode in the second foot pressure distributions PB; obtain a trigger speed V_{P} of the first foot pressure distributions PA and the second foot pressure distributions PB in the first trigger mode pressure distributions PA′ and the second trigger mode pressure distributions PB′; based on the beat speed V_{T1} being slower than the trigger speed V_{P}, increase the beat speed V_{T1}; and based on the beat speed V_{T1} being faster than the trigger speed V_{P}, reduce the beat speed V_{T1}. As a result, the rehabilitation device 100 may obtain the beat speed V_{T1} that matches the rehabilitation action of the rehabilitation person. The rehabilitation person performs rehabilitation at the matching beat speed V_{T1}, which is helpful for the rehabilitation of the rehabilitation person. In addition, the rehabilitation with music may improve the stickiness and interest of rehabilitation and increase the motivation of the rehabilitation person.

The rehabilitation person suffers, for example, from degenerative neurological diseases or other types of diseases. As illustrated in FIG. 2, the rehabilitation person may perform rehabilitation action during the rehabilitation process. The aforementioned rehabilitation action is, for example, walking. During the rehabilitation process, the first pressure sensing device 120A captures a plurality of the first foot pressure distributions PA, the second pressure sensing device 120B captures a plurality of the second foot pressure distributions PB, and the processor 130 analyzes the first foot pressure distributions PA and the second foot pressure distributions PB are configured to obtain the beat speed V_{T1} of the rhythm music T1 that matches the rehabilitation action of the rehabilitation person. In the embodiment of the present disclosure, the process of obtaining the matching beat speed V_{T1} is automatically completed by the rehabilitation device 100.

In another embodiment, the number of the pressure sensing devices of the rehabilitation device 100 may be one. For example, the rehabilitation device 100 may omit the second pressure sensing device 120B, and the processor 130 analyzes the first foot pressure distributions PA to obtain the beat speed V_{T1} of the rhythm music T1 that matches the rehabilitation action of the rehabilitation person. For another example, the rehabilitation device 100 may omit the first pressure sensing device 120A, and the processor 130 analyzes the second foot pressure distributions PB to obtain the beat speed V_{T1} of the rhythm music T1 that matches the rehabilitation action of the rehabilitation person.

As illustrated in FIG. 3A, one of the first foot pressure distribution PA and the second foot pressure distribution PB is, for example, the foot pressure distribution of a left foot of the rehabilitation person, and another of the first foot pressure distribution PA and the second foot pressure distribution PB is, for example, the foot pressure distribution of a right foot of the rehabilitation person. In the present embodiment, the first foot pressure distribution PA is, for example, the foot pressure distribution of the left foot of the rehabilitation person, and the second foot pressure distribution PB is, for example, the foot pressure distribution of the right foot of the rehabilitation person.

As illustrated in FIG. 3A, the first foot pressure distribution PA includes a plurality of the first pressure sensing values PAv arranged in an array. The first foot pressure distribution PA includes an area A1 and an area A2, wherein the area A1 is an area where pressure may be detected (for example, the first pressure sensing value PAv is not 0), such as an area in contact with the sole of the foot, and area A2 is an area where no pressure is detected (for example, the first pressure sensing value PAv is equal to 0), for example, an area where there is no contact with the sole of the foot. The second foot pressure distribution PB includes a plurality of the second pressure sensing values PBv arranged in an array. The second foot pressure distribution PB includes an area B1 and an area B2, where the area B1 is an area where pressure may be detected (for example, the second pressure sensing value PBv is not 0), such as an area in contact with the sole of the foot, and the area B2 is an area where no pressure is detected (for example, the second pressure sensing value PBv is equal to 0), for example, an area where there is no contact with the sole of the foot.

The aforementioned “trigger mode” is, for example, the foot pressure distribution (i.e., trigger mode pressure distribution) corresponding to the maximum extreme value of a plurality of discriminant indexes (e.g., pressure average) of a plurality of the foot pressure distributions. For example, the processor 130 is configured to obtain a first pressure average value of all first pressure sensing values PAv in the area A1 of each first foot pressure distribution PA; obtain a plurality of first maximum extreme values PAₘₐₓ (the first maximum extreme value PAₘₐₓ is illustrated in FIG. 4 corresponding to the first trigger mode pressure distributions PA′) in the first pressure average values of the first foot pressure distributions PA; obtain a plurality of second maximum extreme values PBₘₐₓ (the second maximum extreme value PBₘₐₓ is illustrated in FIG. 4 corresponding to the second trigger mode pressure distributions PB′) in the second pressure average values of the second foot pressure distributions PB; and obtain the trigger speed V_{P} according to the first maximum extreme values PAₘₐₓ and the second maximum extreme values PBₘₐₓ. The trigger speed V_{P} is, for example, the number of the maximum extreme values (the first maximum extreme value PAₘₐₓ and the second maximum extreme value PBₘₐₓ) per unit time (for example, 1 minute), and the crossed symbol in FIG. 4 is the beat points T11 of the rhythm music T1, the unit (for example, BPM (Beats Per Minute)) of the beat speed V_{T1} is, for example, the number of the beat points per unit time (for example, 1 minute).

As illustrated in FIG. 4, the processor 130 constructs a first curve CA using a plurality of the first pressure average values, and constructs a second curve CB using a plurality of second pressure average values. When the rehabilitation device 100 omits the first pressure sensing device 120A, the processor 130 obtains the trigger speed V_{P} by using the second maximum extreme values PBₘₐₓ, and the first curve CA is omitted in FIG. 4. When the rehabilitation device 100 omits the second pressure sensing device 120B, the processor 130 obtains the trigger speed V_{P} by using the first maximum extreme values PAₘₐₓ, and the second curve CB is omitted in FIG. 4.

The aforementioned “trigger mode” is, for example, the foot pressure distribution (i.e., trigger mode pressure distribution) corresponding to the maximum extreme value of a plurality of discriminant indexes (e.g., pressure sum value) of a plurality of the foot pressure distributions. For example, the processor 130 is configured to obtain the first pressure sum value of all first pressure sensing values PAv in the area A1 of each first foot pressure distribution PA; obtain a plurality of first maximum extreme values PAₘₐₓ in the first pressure sum values of the first foot pressure distributions PA; obtain a plurality of second maximum extreme values PBₘₐₓ in the second pressure sum values of the second foot pressure distributions PB; and obtain the trigger speed V_{P} according to the first maximum extreme values PAₘₐₓ and the second maximum extreme values PBₘₐₓ.

As illustrated in FIG. 4, the beat point T11 of the rhythm music T1 substantially coincides with (or matches) the first maximum extreme value PAₘₐₓ (that is, corresponding to the first trigger mode pressure distributions PA′) of the first curve CA and the second maximum extreme value PBₘₐₓ (that is, corresponding to the second trigger mode pressure distributions PB′) of the second curve CB, and it indicates that the beat speed V_{T1} in FIG. 4 matches the rehabilitation action of the rehabilitation person, which is contribute to the recovery of the rehabilitation person.

“Matching” inhere means: the first maximum extreme value PAₘₐₓ of the first curve CA coincide with the beat point T11 of the rhythm music T1 at substantially the same time point, and the second maximum extreme value PBₘₐₓ of the second curve CB coincides with the beat point T11 of the rhythm music T1 at substantially the same time point. The closer each maximum extreme value is to the corresponding beat point T11 in time is, the better the match between the beat speed V_{T1} and the rehabilitation action is.

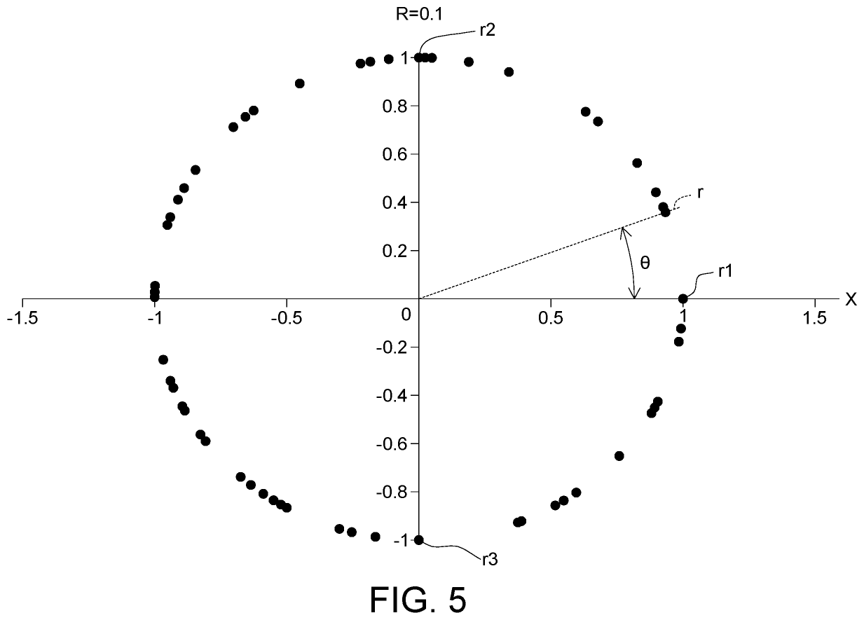

In another embodiment, the processor 130 may adjust the beat speed V_{T1} according to a sequence relationship between the maximum extreme values and the occurrence time points of the beat point T11. The following is illustrated with an example in FIG. 5.

Referring to FIG. 5, FIG. 5 illustrates a schematic diagram of the maximum extreme values and the beat point T11 represented by R-score map according to an embodiment of the present invention. The processor 130 may construct the R-score map according to a plurality of the maximum extreme values (corresponding to the trigger mode pressure distribution) and the occurrence time points of a plurality of the beat points T11 of the rhythm music T1, wherein the R-score map includes a plurality of score points r, each score point r may represent the sequence relationship between the corresponding beat point T11 and occurrence time point of the maximum extreme value. The processor 130 may output the R-score map, allowing the rehabilitation person to understand the rehabilitation situation more intuitively through the R-score chart.

As illustrated in FIG. 5, when the beat point T11 of the rhythm music T1 coincides with the first maximum extreme value PAₘₐₓ of the first curve CA, a score point r1 may be marked in FIG. 5 (the score is equal to 1), the match between the first maximum extreme value PAₘₐₓ and the best point T11 is best. When the first maximum extreme value PAₘₐₓ is slower than the beat point T11 of the rhythm music T1, a score point may be marked in the first quadrant in FIG. 5. For example, a score point r2 indicates that the first maximum extreme value PAₘₐₓ is half a beat slower than the beat point T11 of the rhythm music T1. When the first maximum extreme value PAₘₐₓ is faster than the beat point T11 of the rhythm music T1, a score point may be marked in the fourth quadrant in FIG. 5. For example, a score point r3 indicates that the first maximum extreme value PAₘₐₓ is half the beat faster than the beat point T11 of the rhythm music T1. The score points in the first and fourth quadrants in FIG. 5 have high matching, wherein when the score point r is closer to the beat speed V_{T1} of the horizontal axis, the matching is higher, while the score points located at the second and third quadrants in FIG. 5 have lower matching.

Similarly, as illustrated in FIG. 5, when the beat point T11 of the rhythm music T1 coincides with the second maximum extreme value PBₘₐₓ of the curve CA, the score point r1 (the score is equal to 1) may be marked in FIG. 5, and the matching between the second maximum extreme value PBₘₐₓ and the beat point T11 is optimal. When the second maximum extreme value PBₘₐₓ is slower than the beat point T11 of the rhythm music T1, the score point may be marked in the first quadrant of FIG. 5. For example, the score point r2 means that the second maximum extreme value PBₘₐₓ is slower than the beat point T11 of the rhythm music T1 by half the beat point. When the second maximum extreme value PBₘₐₓ is faster than the beat point T11 of the rhythm music T1, the score point may be marked in the fourth quadrant of FIG. 5. For example, the score point r3 means that the second maximum extreme value PBₘₐₓ is faster than the beat point T11 of the rhythm music T1 by half the beat. The score points located at the first quadrant and the fourth quadrant in FIG. 5 belong the high matching, wherein when the beat speed V_{T1} whose the score point is closer the horizontal axis, the matching is higher, while the score points 20 located at the second quadrant and the third quadrant in FIG. 5 belong the low matching.

After the processor 130 obtains the R-score map of the rehabilitation person, the processor 130 uses the beat speed V_{T1} corresponding to the score point closest to the horizontal axis as the matching beat speed, and outputs such beat speed V_{T1}, or use the beat speed V_{T1} as the beat speed for subsequent or long-term rehabilitation.

In one rehabilitation test cycle, N beat points (the value of BPM is not limited) are tested at the beat speed V_{T1}, wherein N is, for example, a positive integer greater than 1, such as 20; however, the value of N may also be less or more. The processor 130 establishes the R-score map with the 20 sets of score points r after the ten first maximum extreme values PAₘₐₓ in the curve CA and the ten second maximum extreme values PBₘₐₓ in the curve CB are obtained. The processor 130 determines whether an angle θ between each score point r and the horizontal axis X is equal to or less than a preset value according to the R-score map; if the angle θ between each score point r and the horizontal axis value equal to or less than the preset value, it means that the beat speed V_{T1} matches the rehabilitation action; if the angle θ between each score point r and the horizontal axis is less than the preset value, it means that the beat speed V_{T1} does not match the rehabilitation action, and in the next test cycle, the processor 130 may adjust the beat speed V_{T1} of the previous test cycle and repeat the same steps. As a result, a plurality of the rehabilitation test cycles are repeated until the beat speed V_{T1} that matches the rehabilitation action is obtained. Alternatively, the processor 130 performs a plurality of the rehabilitation test cycles and obtains the beat speed V_{T1} corresponding to the minimum angle θ. In addition, the embodiment of the present disclosure does not limit the aforementioned default value.

In another embodiment, during one rehabilitation cycle, the processor 130 may real-timely and dynamically adjust the beat speed V_{T1} according to the trigger speed V_{P}, so that the rehabilitation action of the rehabilitation person keeps matching the beat speed V_{T1}. In addition, the processor 130 may realize the aforementioned dynamic adjustment of the beat speed V_{T1} by using, for example, Kuramoto technology.

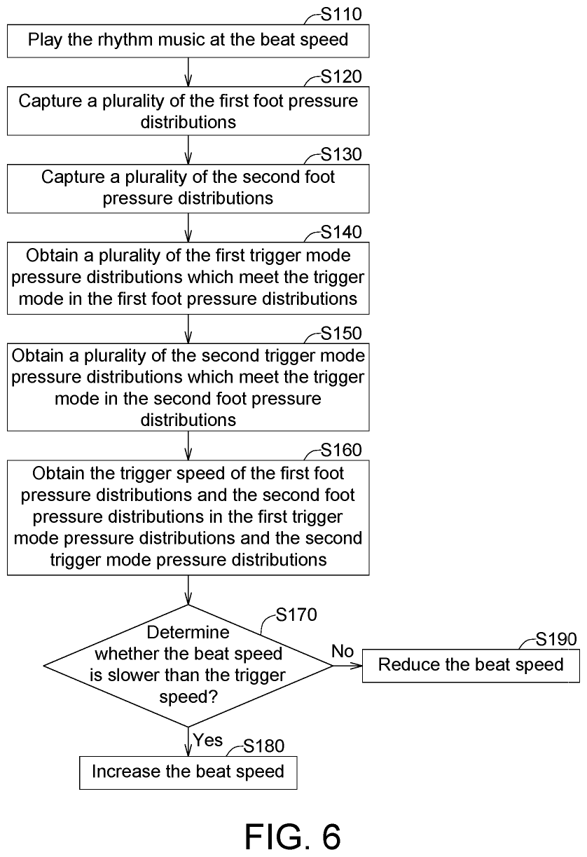

Referring to FIG. 6, FIG. 6 illustrates a flow chart of the rehabilitation method of the rehabilitation device 100 in FIG. 1.

In step S110, the music player 110 plays the rhythm music T1 at the beat speed V_{T1}.

In step S120, the first pressure sensing device 120A captures a plurality of the first foot pressure distributions PA.

In step S130, the second pressure sensing device 120B captures a plurality of the second foot pressure distributions PB.

In step S140, the processor 130 obtains a plurality of the first trigger mode pressure distributions PA′ which meet the trigger mode in the first foot pressure distributions PA.

In step S150, the processor 130 obtains a plurality of the second trigger mode pressure distributions PB′ which meet the trigger mode in the second foot pressure distributions PB.

In step S160, the processor 130 obtains the trigger speed V_{P} of the first foot pressure distributions PA and the second foot pressure distributions PB in the first trigger mode pressure distributions PA′ and the second trigger mode pressure distributions PB′.

In step S170, the processor 130 determines whether the beat speed V_{T1} is slower than the trigger speed V_{P}. If the beat speed V_{T1} is slower than the trigger speed V_{P}, the process proceeds to step S180; if the beat speed V_{T1} is faster than the trigger speed V_{P}, the process proceeds to step S170.

In step S180, the processor 130 increases the beat speed V_{T1} to increase the matching between the beat speed V_{T1} and the rehabilitation action of the rehabilitation person.

In step S190, the processor 130 reduces the beat speed V_{T1} to increase the matching between the beat speed V_{T1} and the rehabilitation action of the rehabilitation person.

In summary, embodiments of the present disclosure propose a rehabilitation method and a rehabilitation device using the same, using plantar pressure analysis technology to obtain a plurality of the trigger mode pressure distributions which meet the trigger mode in a plurality of the foot pressure distributions, and obtain the trigger speed of the trigger mode pressure distributions. In an embodiment, the rehabilitation device is further configured to: increase the beat speed based on the beat speed of the rhythm music being slower than the trigger speed; and reduce the beat speed based on the beat speed of the rhythm music being faster than the trigger speed. As a result, by adjusting the beat speed, the beat speed that matches the rehabilitation action may be obtained. In other embodiment, the rehabilitation device may adjust the beat speed according to the sequence relationship between the trigger mode pressure distribution and the occurrence time point of the beat point of the rhythm music by using, for example, the R-score method.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A rehabilitation method, including:
playing a rhythm music at a beat speed by a music player;
capturing a plurality of first foot pressure distributions by a first pressure sensing device;
obtaining a plurality of first trigger mode pressure distributions which meet a trigger mode in the first foot pressure distributions by a processor;
obtaining a trigger speed of the first trigger mode pressure distributions in the first foot pressure distributions by the processor;
increasing the beat speed based on the beat speed being slower than the trigger speed by the processor; and
reducing the beat speed based on the beat speed being faster than the trigger speed by the processor.

2. The rehabilitation method according to claim 1, further comprising:
constructing a R-score map according to the first trigger mode pressure distributions and a plurality of occurrence time points of a plurality of beat points of the rhythm music, wherein the R-score map comprises a plurality of score points;
outputting the beat speed based on an angle between each score point and a horizontal axis in the R-score map being equal to or less than a preset value.

3. The rehabilitation method according to claim 1, further comprising:
capturing a plurality of second foot pressure distributions by a second pressure sensing device;
obtaining a plurality of second trigger mode pressure distributions which meet the trigger mode in the second foot pressure distributions by the processor; and
obtaining the trigger speed of the first trigger mode pressure distributions and the second trigger mode pressure distributions in the first foot pressure distributions and the second foot pressure distributions.

4. The rehabilitation method according to claim 1, further comprising:
obtaining a first pressure average of a plurality of first pressure sensing values in each first foot pressure distribution by the processor; and
obtaining a plurality of first maximum extreme values in the first pressure averages by the processor; and
obtaining the trigger speed according to the first maximum extreme values by the processor.

5. The rehabilitation method according to claim 1, further comprising:
obtaining a first pressure sum value of a plurality of first pressure sensing values in each first foot pressure distribution by the processor; and
obtaining a plurality of first maximum extreme values in the first pressure sum value by the processor; and
obtaining the trigger speed according to the first maximum extreme values by the processor.

6. A rehabilitation device, comprising:
a music player configured to play a rhythm music at a beat speed;
a first pressure sensing device configured to capture a plurality of first foot pressure distributions; and
a processor configured to:
obtain a plurality of first trigger mode pressure distributions which meet a trigger mode in the first foot pressure distributions;
obtain a trigger speed of the first trigger mode pressure distribution in the first foot pressure distributions;
increase the beat speed based on the beat speed being slower than the trigger speed; and
reduce the beat speed based on the beat speed being faster than the trigger speed.

7. The rehabilitation device according to claim 6, wherein the processor is further configured to:
construct a R-score map according to the first trigger mode pressure distributions and a plurality of occurrence time points of a plurality of beat points of the rhythm music, wherein the R-score map comprises a plurality of score points; and
output the beat speed based on an angle between each score point and a horizontal axis in the R-score map being equal to or less than a preset value.

8. The rehabilitation device according to claim 6, further comprising:
a second pressure sensing device configured to capture a plurality of second foot pressure distributions;
wherein the processor is configured to:
obtain a plurality of second trigger mode pressure distributions which meet the trigger mode in the second foot pressure distributions; and
obtain the trigger speed of the first trigger mode pressure distributions and the second trigger mode pressure distributions in the first foot pressure distributions and the second foot pressure distributions.

9. The rehabilitation device according to claim 6, wherein the processor is further configured to:
obtain a first pressure average of a plurality of first pressure sensing values in each first foot pressure distribution; and
obtain a plurality of first maximum extreme values in the first pressure average values; and
obtain the trigger speed according to the first maximum extreme values.

10. The rehabilitation device according to claim 6, wherein the processor is further configured to:
obtain a first pressure sum value of a plurality of first pressure sensing values in each first foot pressure distribution; and
obtain a plurality of first maximum extreme values in the first pressure sum value; and
obtain the trigger speed according to the first maximum extreme values.
